(19) United States
(12) Patent Application Publication
(10) Pub. No.: US 2007/0087020 A1
(43) Pub. Date: Apr. 19, 2007

(54) Use of parasitic biological agents for prevention and control of allergic and other IgE-mediated disorders

(76) Inventor: Daniel J. O'Connor, Princeton, NJ (US)

Correspondence Address: KELLEY DRYE & WARREN LLP, 3050 K STREET, NW, SUITE 400, WASHINGTON, DC, 20007, US

(21) Appl. No.: 11/340,507

(22) Filed: Jan. 27, 2006

Related U.S. Application Data
(63) Continuation-in-part of application No. 11/187,819, filed on Jul. 25, 2005.
(60) Provisional application No. 60/647,032, filed on Jan. 27, 2005.

(51) Int. Cl. A61K 39/002

(52) U.S. Cl. 424/265.1

(57) ABSTRACT

The present invention describes using, on a repetitive basis, a non-human colonizing helminth compound, in an amount sufficient to establish a transitory parasitic helminth infection and or to simulate in a parasitic helminth infection, thereby having immunosuppressive effect against benign antigens and or stimulating a regulatory immune response characterized by the production of T helper cells 2 (Th2), T regulatory helper cells (TREG) and certain cytokines, including, but not limited to interleukin 10 (IL-10), as a therapy or prophylaxis of allergy and other IgE-mediated disorders, which are marked by an inappropriate IgE immune response including, but not limited to an aberrant and or enhanced IgE antibody production to benign antigens. The invention presents using helminth compound by administering it in a frequency and amount sufficient to eliminate or ameliorate the inappropriate immune response in an asthmatic and or allergic individual.

### CROSS REFERENCE TO RELATED APPLICATIONS

The present invention relates to, and is entitled to the benefit of the earlier filing date and priority of, Application No. 60/647,032, filed on Jan. 27, 2005, and is a continuation-in-part of application Ser. No. 11/187,819, filed Jul. 25, 2005, both of which are herein incorporated by reference as if fully set forth.

### FIELD OF THE INVENTION

This invention relates to the treatment of disease states in a parasite-free individual that is marked by enhanced and/or aberrant IgE immune responses to benign antigens, including an inappropriate Th2 cell production.

### BACKGROUND OF THE INVENTION

Helminths are elaborate multicellular worms with complex life cycles and development. Helminths inhabit their host's intestines and, in order to survive, establish a relationship with the host's mucosal defenses. The nematodes (nonsegmented roundworms) and the platyhelmiuths (flatworms) are the two groups of helminths that inhabit the human intestines.

Helminth infections are highly prevalent in the human population, particularly in tropical and subtropical countries. Helminths that are classified as parasites that infect humans are so classified because they are known to have a pathological effect on the human host. Helminths with no associated pathology in humans are known as comiciles. Twenty-six species of helminth parasites have been reported to infect humans. Among these parasites, nematode species that colonize the gastrointestinal tract are of concern in terms of overall morbidity. The four most prevalent species of nematodes—Ascaris lumbricoides, Trichuris trichiura, Necator americanus and Ancylostoma duodenale—infect more than a billion people worldwide (Chan, M-S. The global burden of intestinal nematode infections. Fifty years on. Parasit. Today, 13: 438-443, 1997). The prevalence of helminths is highest in rural and underdeveloped areas characterized by overcrowding, poor sanitation and an impure food/water supply. When compared to areas where the standard of living is higher, asthma and allergies occur at a much lower rate in these rural and underdeveloped regions.

Allergy and asthma are diseases marked by inappropriate immune system responses to benign, allergy causing substances, such as pet dander, dust mites and pollen. Allergy and asthma can cause airways to become blocked or narrowed. Generally, they cause a shortness of breath, wheezing, coughing, breathing trouble and other symptoms. If an asthma or allergic attack is severe, the person may need emergency treatment to restore normal breathing. In certain cases, this inappropriate immune system response is so overwhelming that it can be fatal.

In the United States, there are more than 50 million people, approximately twenty percent of the population, who suffer from the allergic diseases of asthma and or allergies (Safety & Health Policy Center, A Division of the National Safety Council, 1025 Connecticut Avenue, NW, Suite 1200, Washington, D.C. 20036. Apr. 8, 2004). Americans spend at least $5 billion a year in an attempt to cope with these afflictions. In the United States, asthma is the most common chronic disease of childhood. The number of young people and children with asthma is rising. About 17 million Americans have asthma and almost 9 million children have been diagnosed with asthma at some point in their life. Nearly one in 13 school-aged children has asthma. Between 1980-1994, asthma among children under five years old increased by 160 percent. Nearly one in five of all pediatric emergency room visits is asthma-related. This is an increase of approximately 45 percent in the past decade. Asthma attacks in children contribute to parents making nearly a million emergency room visits every year, which accounts for half of the $2 billion cost of treating children with the illness.

In the United States, asthma causes approximately 5,000 deaths per year. Peanut allergies account for 50 to 100 deaths in the United States each year (CNN, Education, Friday, Jan. 30, 2004). Although most asthmatics that die of the disease are more than 50 years old, rates of asthma death have increased in almost all age groups. Most asthma deaths occur in urban areas. Worldwide, the prevalence of asthma has increased and continues to increase dramatically (R. Beasley, Global Burden of Asthma. (Commissioned by Global Initiate for Asthma (GINA). Data obtained on the burden of asthma in 20 different regions worldwide from literature primarily published through the International Study of Asthma and Allergies in Childhood (ISAAC) and the European Community Respiratory Health Survey (ECHRS). The Global Initiative for Asthma (GINA) program was initiated with the U.S. National Heart, Lung, and Blood Institute, NIH and the World Health Organization (WHO) in an effort to raise awareness among public health and government officials, health care workers, and the general public that asthma was on the increase.)). The international patterns of asthma prevalence are not explained by the current knowledge of the causation of asthma. Asthma has become more common in both children and adults around the world in recent decades. The increase in the prevalence of asthma has been associated with an increase in atopic sensitization, and is paralleled by similar increases in other allergic disorders such as eczema and rhinitis.

It is also estimated that worldwide as many as 300 million people of all ages, and all ethnic backgrounds, suffer from asthma and the burden of this disease to governments, health care systems, families, and patients is increasing worldwide. Asthma is one of the most common chronic diseases in the world. With the projected increase in the proportion of the world's population dwelling in urban areas from 45% to 59% by 2025, there is likely to be a marked increase in the number of asthmatics worldwide over the next two decades. Some sources estimate that there may be an additional 100 million persons with asthma by 2025. This trend has been building for decades. In fact, there has been a significant global increase in the prevalence of allergic diseases over the past 40 years (R. Beasley, Global Burden of Asthma).

There is a considerably lower prevalence of allergic diseases in developing countries (The International Study of Asthma and Allergies in Childhood Steering Committee, Lancet 351, 1225 (1998)). There are also clear differences in the prevalence of allergies between rural and urban areas within one country. For example, in Ethiopia, asthma is more prevalent in urban areas than in rural villages (H. Yemaneberhan et al., Lancet 350, 85 (1997)), and asthma is more common in residents of urban Germany than in farmers living in rural Bavaria (O. S. von Ehrenstein et al., Clin. Exp. Allergy 30, 187 (2000)).

Because allergic diseases are most prevalent in highly industrialized regions and the relative absence of allergic disease in underdeveloped regions, it has been suggested that there is some critical environmental factor responsible for the increasing frequency of asthma and allergies in regions as they develop. People in industrialized regions live in increasingly hygienic environments and, as a result, acquire helminths much less frequently than those people living in rural areas. The increase of asthma and allergic diseases in the industrialized world has also been explained by a decline in bacterial and viral infections during childhood. This explanation, commonly known as the “Hygiene Hypothesis,” is explained from an immunological perspective as bacterial and viral infections during early life direct the maturing immune system toward the functional T-helper 1 cells (Th1) which counterbalance pro-allergic responses of T-helper 2 cells (Th2) (Strachan, D. P., Br. Med. J. 299:1295 (1989)). Thus, a reduction in the overall microbial burden is thought to result in a weak Th1 imprinting and unrestrained Th2 responses that allow an increase in allergy (Cookson, W. O. C. M. and Moffatt, M. F., Science, 275:41(1997)).

This theory is contradicted by observations that the prevalence of Th1-autoimmune diseases, such as Crohn's disease, are also increasing and that Th2-skewed helminth infections are disassociated with allergy and asthma. Further, this theory fails to consider that the worldwide trend toward modernization and, as a consequence, greater hygiene (i.e., food and water purification, increased sanitation efforts and medicines to treat parasitical infections), may be resulting in a kind of worldwide “de-worming”. In other words, the elimination of the chronic immune system stimulation induced by a helminthic infection that has occurred in recent times may account for the increase in asthma and allergies.

The potential connection between asthma, allergies and parasites is not new. In the 1960's and 1970's there was vigorous scientific debate around the idea that helminths provide a protective effect against allergies. In addition to anecdotal reports of protection from hay fever by ingestion of Ascaris spp. (roundworm) eggs (J. A. Turton, Lancet 2, 686 (1976)), a meta-analysis of data from early surveys showed that, despite the variation in methodology and clinical assessment of allergies, the prevalence of parasitic helminth infections was negatively associated with the prevalence of asthma (S. Masters, E. Barrett-Connor, Epidemiol. Rev. 7, 49 (1985)).

Humans, as parasitic helminth hosts, can acquire various helminthic species through contact with soil, food or water contaminated with the infective form of the parasite. Children in rural areas of the world most frequently harbor parasitic helminth infections because of their close contact with soil and less than optimal hygienic practices. When a parasitic helminth enters the body through begins to shed proteins, the body's immune mechanism is activated. In short, the body's immune response to a parasitic helminth causes the production of billions of Y-shaped antibodies to the foreign proteins shed by the helminth. Lymphocytes are one of the five kinds of white blood cells or leukocytes. There are several kinds of lymphocytes, each with different functions to perform. The most common types of lymphocytes are B lymphocytes or B cells, which are responsible for making antibodies.

The gastrointestinal (GI) tract is an organ for digestion, absorption and excretion. It is one of the largest immunological organs of the body, and it serves as the first line of defense against intestinal pathogens (e.g., bacteria, parasites). Gut-associated lymphoid tissues (GALT) make up ˜25% by weight of the gut mucosa and submucosa and thus constitute the largest extrathymic site of lymphocytes in humans (McBurney M. I. The gut: central organ in nutrient requirements and metabolism. Can. J. Physiol. Pharmacol. 1993;72:260-265). Cells in GALT respond to intestinal pathogens by processing antigens for recognition by lymphocytes, by initiating a cascade of specialized immune responses to the antigens, by regulating the migration of immune mediators from the periphery to the infected gut and by participating directly in cytotoxic activities that limit parasite establishment and survival. In addition to these specific immunological responses, the GI tract performs nonspecific barrier functions (Van der Hulst R. R. W. J., Von Meyenfeldt M. F., Van Kreel B. K., Thunnissen F. B. J. M., Brummer R.-J. M., Arends J.-W., Soeters P. B. Gut permeability, intestinal morphology, and nutritional depletion. Nutrition 1998;14:1-6) (Welsh F. K. S., Farmery S. M., MacLennan K., Sheridan M. B., Barclay G. R., Guillou P. J., Reynolds J. V. Gut barrier function in malnourished patients. Gut 1998;42:396-401). Mucus secretion and formation of tight cell junctions prevent the entry of bacteria and other pathogenic antigens, and rapid mucosal turnover enables the repair of epithelial or lymphoid cells damaged by parasitic infections.

The gut mucosal immune system consists of two anatomically and functionally distinct compartments: (1) the specialized local inductive sites (Peyer's patches, isolated lymphoid follicles, mesenteric lymph nodes), where intestinal antigens are first recognized; and (2) diffuse effector sites (intraepithelium and lamina propria), where the outcome of an effective immune response is elimination of the infectious agent. Luminal antigens are transported across epithelial barriers either by specialized epithelial M cells or by intraepithelial lymphocytes (mostly T cells) to the organized lymphoid tissues within the mucosa (e.g., Peyer's patches). After epithelial transport, antigens are processed and presented by antigen-presenting cells (APC) such as dendritic cells, B cells, macrophages and other intestinal epithelial cells. Naive T lymphocytes first interact with antigen-primed APC in aggregated Peyer's patches and single lymphoid follicles and then further differentiate in the germinal centers of the lymphoid follicles. Thereafter, the antigen-specific T and B cells leave the epithelial barrier to collect in the mesenteric lymph nodes (MLN), which drain the mucosa and supply the peripheral bloodstream with gut-derived or locally activated immune cells, or both. From the blood, the lymphocytes migrate to systemic lymphoid tissues such as the spleen and peripheral lymph nodes, where the lymphocytes proliferate and mature either into effector lymphocytes, which secrete cytokines and mediate T cell-dependent humoral immunity, or into memory cells that can respond rapidly to the infection on secondary encounter. Peripheral lymphocytes can preferentially leave the blood vessels and move into the intestinal lamina propria and intraepithelium by expressing adhesion receptors that are recognized by specific endothelial molecules lining the gut mucosal lymphoid tissues. As a result, most of the antigen-committed and differentiated lymphocytes that enter the effector sites of GALT are likely to have had prior contact with, and specific activation by, parasite antigens located in the gut mucosa. The continuous migration of lymphocytes from intestinal lymphoid tissues to the bloodstream and back enables the GALT to carry out two important roles in the defense against intestinal parasites. First, it allows delivery of the parasite antigen to peripheral sites, initiating a widely disseminated immune response, and second, it promotes trafficking of gut-derived lymphocytes from the blood to effector sites within the intestinal epithelium. Gut-associated lymphocytes further contribute to host defense against GI parasites by secreting cytokines that regulate the appropriateness, magnitude and phenotypic expression of immune responses.

Lymphocytes are one of the five kinds of white blood cells or leukocytes. There are several kinds of lymphocytes, each with different functions to perform. The most common types of lymphocytes are B lymphocytes or B cells, which are responsible for making antibodies. T lymphocytes or T cells, one of which is T helper cells or Th cells, enhance the production of antibodies by B cells. B cells are specialized white blood cells produced in the bone marrow. Although bone marrow is the ultimate source of lymphocytes, the lymphocytes that will become T cells migrate from the bone marrow to the thymus where they mature. Both B cells and T cells also take up residence in lymph nodes, the spleen and other tissues where they encounter antigens, continue to divide by mitosis, and mature into fully functional cells. Each B cell contains multiple copies of one kind of antibody as a surface receptor for antigens. The entire population of B cells has the ability to specifically bind to millions of different antigens.

Depending on the type of antigenic stimulus, undifferentiated T helper (Th) cells transform into either Th1 or Th2 cells. Th cells also regulate other cells of the immune system through secretion of molecules called cytokines. Cytokines are messenger substances that regulate the immune system. It is believed that the type of cytokine that is secreted determines the nature of the inflammatory response. In other words, the immune responses usually present as either T-helper 1 cells (Th1), which display certain cytokine profiles and which counterbalance pro-allergic responses of T-helper 2 (Th2), which also display polarized cytokine profiles. For example, the excretion of Th1 causes an inflammatory reaction, while the excretion of Th2 brings about an inflammation-inhibiting reaction in the immune system. There are two distinct kinds of T-helper cells, Th1 and Th2. Th1 participates in cell-mediated immunity. They are essential for controlling such intracellular pathogens as viruses and certain bacteria. Th2 provide help for B cells and, in so doing, are essential for antibody-mediated immunity. Bacterial, viral and protozoan infections usually stimulate a Th1 response, characterized by elevated levels of Th1 cytokines (i.e., interleukin (IL)-2, IL-12, interferon (IFN)) and effectors such as macrophages, natural killer cells and neutrophils. In such Th1 responses, cell-mediated immunity involving phagocytosis is responsible for the functional immunity. Th1-type inflammations produce large amounts of IFN-y and tumor necrosis factor (TNF)-alpha. In contrast, the immune response to intestinal parasitic helminth depends on the production of Th2 cytokines (e.g., IL-4, IL-5, IL-10), which mediate antibody-dependent effector responses described below. These Th2 cytokines released in the GALT attract progenitors of B cells, mucosal mast cells (MMC) and eosinophils by chemotaxis to the mucosal epithelium, where they proliferate and mature in response to the stimulatory signals of cytokines and parasite antigens.

Antibodies are needed to control extracellular pathogens, such as parasites, which are exposed to antibodies in blood, other body fluids and the GI tract. There are five different types of antibodies found in humans. The type of antibody released to combat a parasite, immunoglobulin E, or IgE, is least common of the five. When released, the IgE antibody attaches the lower portion of its Y-shape onto the surface of MMC. MMC, each which contain thousands of histamine packed globular granules, are found in high concentrations in human skin, in the membranes of the eyes, nose, and throat, and in the lining of the lungs and gut.

Each MMC has hundreds of thousands of Y-shaped antibodies protruding from its surface. When a protein shed by a parasite sticks (or cross-links) between the arms of two adjacent IgE antibodies, a chain reaction occurs that ends with the MMC releasing its thousands of globular granules. The granules, in turn, release their stored histamines, along with other chemicals, which infiltrate the skin and other tissues close to the activated MMC. These chemicals cause all of the symptoms of inflammation, namely itching, dilated and leaky blood vessels, swelling and excess mucus secretion, all of which are effective in isolating and eliminating the parasite before it can multiply. This immune response protects the body.

Although parasitic helminthes induced a polarized Th2 response, as described above, they have been shown to confer protections against allergies and asthma. Recent studies have reevaluated findings in South America and Africa using a combination of parameters to assess allergy with careful parasitological diagnosis and have shown a consistent inverse relation between helminth infections (schistosomiasis and intestinal helminths) and either skin reactivity to environmental allergens or clinical scores, such as airway hyper-responsiveness, wheeze, and asthma (S. Masters, E. Barrett-Connor, Epidemiol. Rev. 7, 49 (1985)). In most of these studies, 30% of the studied subjects carried substantial levels of IgE to house dust mite (HDM-lgE); these values correspond to those seen in many industrialized countries. In high-income countries, allergen-specific IgE leads to skin reactivity to mite, but in less developed countries, the presence of specific IgE does not always translate into equivalent numbers of atopic (skin) reactions. In Gabon, only 11% of the school children reacted to mite in a skin prick test (SPT), whereas 32% were positive for HDM IgE (A. H. J. van den Biggelaar et al., Lancet 356, 1723(2000).20. O. A. Nyan et al., Clin. Exp. Allergy 31, 1672 (2001)). High levels of IgE and SPT positivity in affluent societies in central Europe 33%) (J. Riedler et al., Lancet 358, 1129 (2001)), and Australia (32.5%) (A. O. Faniran, J. K. Peat, A. J. Woolcock, Thorax 54,606 (1999)) are associated with high prevalences of airway disease (12% asthma in central Europe and 21.9% wheeze in Australia).

By contrast, in many low-income countries, such as Gambia (O. A. Nyan et al., Clin. Exp. Allergy 31, 1672 (2001)) and Nigeria (A. O. Faniran, J. K. Peat, A. J. Woolcock, Thorax 54,606 (1999)), 35.3% and 28.2% atopic reactions translated into only 3.6% asthma and 6% wheeze, respectively. In a recent study in Ethiopia, atopy to HDM was common, but in the presence of high intensity intestinal helminth infections was unrelated to wheeze (S. Scrivener et al., Lancet 358, 1493 (2001)). Thus, despite IgE sensitization to environmental allergens, helminth-infested subjects are somehow protected from mast cell degranulation and inflammatory responses in affected organs.

Researchers found that Ethiopians who showed signs of having had hookworm infestation were far less likely to report asthmatic symptoms. In a study conducted at the University of Nottingham and Jimma University in Ethiopia (Am J Respir Crit Care Med. 2003 May 15;167(10):1369-73), researchers examined over 200 Ethiopians with asthma, and compared them with almost 400 non-asthmatic Ethiopians. Fecal samples were examined for signs of parasitic infection. In total, hookworm was present in 24% of everyone tested and people with hookworm infestation were only half as likely to have asthmatic symptoms and there was a relationship between the level of hookworm infestation and the prevalence of asthma. The study concluded that there was increased risk for asthma in urban areas covered by the project partly due to the protective effect of hookworm infestation.

The amount of and chronic nature of helminth infections may be an important variable that may determine whether helminths act as a risk factor for, or confer protection against, allergic diseases. In Venezuela, the classification of helminth-infested populations into those with none, light, or heavy worm burdens shows that light helminth infections are associated with the amplification of allergen-specific IgE responses and high skin reactivity, whereas heavily parasitized subjects are protected from atopic skin reactivity despite a high degree of sensitization to mite (S. Masters, E. Barrett-Connor, Epidemiol. Rev. 7, 49 (1985)). Divergent outcome of Th2 responses in industrialized (low pathogen exposure) and developing countries (high pathogen exposure).

As stated above, a Th2-type response of inflammation commonly seen in allergic reactions is the same Th2 immune response that the body mounts against parasitic helminths. In other words, because external elements, such as dust mites, pollen and peanuts, are inappropriately determined by the immune system of allergic people to be allergens, they are met with the same IgE immune response as the body mounts against a parasitic helminth infection.

When the human body first encounters an allergen like ragweed pollen, the pollen's foreign proteins activate the antibody-dependant effector responses, i.e., the Th2 branch of the immune system, and IgE antibodies are quickly posted on MMC in vulnerable pollen-exposed areas, such as the nose, respiratory tract, and eyes. When the body encounters ragweed proteins again, the IgE antibodies on the MMC catch the foreign proteins, and release the mast cells spew forth histamine, the histamine causes inflammation, and the body experiences the familiar symptoms of hay fever, namely a runny nose, sneezing, coughing, and itchy, watery eyes. Similarly, if the foreign proteins are dust mites that find their way to the lungs, the allergic reaction can trigger the wheezing and shortness of breath associated with asthma. In the same way, a meal of shellfish can produce the upset stomach and diarrhea of food allergy.

Allergists do not know why the human immune system attacks such benign substances as pet dander, dust mites and pollen, reacting as if they were parasites. However, allergists do know that IgE-mediated disorders, including asthma, food allergies, hypersensitivity and anaphylactic reactions are unlike any other immune reaction, except for one, the immune system's response to parasites. The difference between a parasite and ragweed, dust mites or peanuts, is that parasites can be fatal if the IgE antibodies do not kill them first. Allergens such as ragweed, dust mites or peanuts, however, in a non-allergic individual are harmless to the body.

The mechanisms by which immune responses to nonpathogenic environmental antigens lead to either allergy or nonharmful immunity are unknown; however, it has been theorized that the immune responses in healthy and allergic individuals are characterized by a fine balance between allergen-specific T Regulatory 1 cells (TREG) and Th2 cells (J Exp Med. 2004 Jun. 7;199(11):1567-75. Epub 2004 Jun. 01). Single allergen-specific T cells constitute a very small fraction of the whole CD4(+) T cell repertoire and can be isolated from the peripheral blood of humans according to their cytokine profile. Freshly purified interferon-gamma-, IL4, and IL-10-producing allergen-specific CD4(+) T cells display characteristics of Th1, Th2, and T regulatory (TREG) like cells, respectively. TREG cells consistently represent the dominant subset specific for common environmental allergens in healthy individuals; in contrast, there is a high frequency of allergen-specific IL-4-secreting T cells in allergic individuals. TREG cells use multiple suppressive mechanisms, IL-10 and TGF-beta as secreted cytokines, and cytotoxic T lymphocyte antigen 4 and programmed death 1 as surface molecules. Healthy and allergic individuals exhibit all three allergen-specific subsets in different proportions, indicating that a change in the dominant subset may lead to allergy development or recovery. Accordingly, blocking the suppressor activity of TREG cells or increasing Th2 cell frequency enhances allergen-specific Th2 cell activation ex vivo. These results indicate that the balance between allergen-specific TREG cells and Th2 cells may be decisive in the development of allergy.

In a recent study at the Mucosal Immunology Laboratory at Massachusetts General Hospital and Harvard Medical School the influence of a helminth infection on peanut allergy was examined (Upon peanut (PN) antigen (Ag) challenge, mice fed PN extract plus the mucosal adjuvant cholera toxin (CT) produced PN-specific IgE that correlated with systemic anaphylactic symptoms and elevated plasma histamine. PN-specific IgE was not induced in helminth-infected mice fed PN without CT. Moreover, when PN plus CT was fed to helminth-infected mice, both PN-specific IgE and anaphylactic symptoms were greatly diminished. The down-regulation of PN-specific IgE was associated with a marked reduction in the secretion of IL-13 by PN-specific T cells. When helminth-infected PN plus CT-sensitized mice were treated with neutralizing Abs to IL-10, the PN-specific IgE response and anaphylactic symptoms were similar to, or greater than, those seen in mice that receive PN and CT alone). The results of the research illustrated that a helminth infection protects against peanut allergy and that a chronic helminth infection can block the induction of allergen-specific IgE by influencing the behavior of the peanut antigen specific TH cells that are required for this response. The results suggested that helminth-dependent protection against allergic disease involves immunoregulatory mechanisms that block production of allergen-specific IgE (J Immunol. 2002 Sep. 15;169(6):3284-92).

Mechanisms used by parasites to evade the host may include a number of different immunoregulatory mechanisms known in the art as immunosuppression. Immunosuppression is the reduction of the host's immune response either to the parasite specifically or to foreign antigens in general. In other words, immunosuppression can be either specific, i.e., suppression only the host's immune response to the parasite or more general, i.e., involving host's immune response to various nonparasite antigens. A variety of mechanisms have been suggested to explain the immunosuppression, such as: (1) the presence in the infected host of parasite or host substances that nonspecifically stimulate the growth of antibody-producing B cells, rather than stimulating the proliferation of specific antiparasite B-cells; (2) proliferation of suppressor T-cells and/or macrophages that inhibit the immune system by excretion of regulatory cytokines; and (3) production by the parasite of specific immune suppressor substances.

Helminths release a variety of molecules, known in the art as excretory and secretory products (ESP), into the host, which are believe to play a role in host immunosuppression. Although the composition of ESP is largely unknown, it is a source of components for the treatment of allergic disease because ESP is able to induce protection for the helminth from the host's immune response through immunosuppression, and, in doing so, inhibits the induction of allergen-specific IgE by influencing the behavior of the antigen specific TH cells that are required for this response. Stated another way, helminth-dependent protection against allergic disease involves immunoregulatory mechanisms by the product of the helminth of specific immune suppressor substances that blocks production of allergen-specific IgE.

At the Yale University School of Medicine, research was recently conducted that showed that chitin, a surface component of parasites, which induces the production of chitinases in lower life forms during infections with parasites, ameliorated Th2 inflammation and airway hyperresponsiveness and thus may be an important mediator of asthma (Science. 2004 June 11;304(5677):1678-82. Chitin is a surface component of parasites and insects, and chitinases are induced in lower life forms during infections with these agents. Although chitin itself does not exist in humans, chitinases are present in the human genome. Researchers have shown that acidic mammalian chitinase (AMCase) is induced via a Th2-specific, interleukin-13 (IL-13)-mediated pathway in epithelial cells and macrophages in an aeroallergen asthma model and expressed in exaggerated quantities in human asthma. AMCase neutralization ameliorated Th2 inflammation and airway hyperresponsiveness, in part by inhibiting IL-13 pathway activation and chemokine induction. AMCase may thus be an important mediator of IL-13-induced responses in Th2-dominated disorders such as asthma).

An advantage of an embodiment of the present invention is the creation of a biopharmaceutical composition comprised of a pathogen-free non-human colonizing helminth compound, which will have an immunosuppressive effect against benign antigens in an allergic individual. The helminth compound will be administered to an individual with allergic disease in an amount and frequency sufficient to establish a transitory parasitical infection and or to simulate in a parasitic helminth infection, thereby having immunosuppressive effect against benign antigens and or stimulating a regulatory immune response characterized by the production of TREG and certain other cytokines, including IL-10, as a therapy or prophylaxis of allergic and other IgE-mediated disorders, including, but not limited to, asthma, allergies, specifically, CFA's, hypersensitivity and anaphylactic reactions, which are marked by an excessive IgE immune response including an aberrant/enhanced IgE antibody production to benign antigens. An embodiment of the present invention is a method using a helminth compound by administering it in an amount sufficient to eliminate, ameliorate, or reduce the excessive immune response in an asthmatic and or allergic individual. An embodiment of the present invention is generally directed to diseases other IgE-mediated disorders, including asthma, CFA, hypersensitivity and anaphylactic reactions. More specifically, the invention may be directed toward the treatment of peanut allergy, as well as other CFA.

An embodiment of the present invention provides a method of creating an immune environment in an allergic individual that is conducive to ameliorating or eliminating asthma and allergies, specifically, CFA. An embodiment of the present invention relates to a pharmaceutical composition comprising a helminth compound capable of stimulating immunoregulatory mechanisms that blocks production of benign allergen-specific IgE.

Allergic individuals (or hypersensitve individuals) who are exposed to allergens, such as common food products, will react in an allergic manner otherwise described herein. Further, the exposure to these allergens can also result the manifestation of other diseases, specifically the diseases of autism and stuttering. There is growing evidence that many people with autism are sensitive to certain food products. An embodiment of the present invention describes using, on a repetitive basis, a non-human colonizing helminth compound, in an amount sufficient to establish a transitory parasitic helminth infection and or to simulate in a parasitic helminth infection, thereby having immunosuppressive effect against benign antigens and or stimulating a regulatory immune response as a therapy or prophylaxis of allergy and other IgE-mediated disorders. In doing so, the administration of the helminth compound will reverse or ameliorate the allergic sensitivity, thereby also reversing or ameliorating other behavioral and physical conditions, specifically autism and stuttering.

A further embodiment of the present invention provides a pathogen-free non-human colonizing helminth and an acceptable pharmaceutical carrier, which is capable of making a transitory infection in the GI tract without exposing the individual to any harmful effects of attributable to the helminth. Moreover, an embodiment of the present invention provides a method of preparing a pathogen-free non-human colonizing helminth. Furthermore, the present invention relates to providing a helminth compound that will prevent or treat asthma and or allergies specifically, CFA, in an allergic individual.

### SUMMARY OF THE INVENTION

An embodiment of the present invention relates to using, on a repetitive basis, a pathogen-free non-human colonizing helminth compound, in an amount sufficient to establish a transitory parasitic helminth infection and or to simulate a parasitic helminth infection and thereby having immunosuppressive effect against benign antigens and or stimulating a regulatory immune response characterized by the production of T helper cells 2 (Th2), T regulatory helper cells (TREG) and certain cytokines, including, but not limited to interleukin 10 (IL-10), as a therapy or prophylaxis of allergy and other IgE-mediated disorders, which are marked by an inappropriate IgE immune response including, but not limited to an aberrant and or enhanced IgE antibody production to benign antigens. In some embodiments, the invention relates to using helminth compound by administering it in a frequency and amount sufficient to reduce, eliminate or ameliorate the inappropriate immune response in an asthmatic and or allergic individual. This invention is generally directed to diseases and IgE-mediated disorders, including asthma, allergies, hypersensitivity and anaphylactic reactions. More specifically, the present invention is directed toward the treatment of certain CFA.

The helminth compound may comprise a pathogen-free non-human colonizing helminth consisting of a live adult helminth, ground adult helminth, adult helminth extract, adult helminth ESP, live helminth larvae, ground helminth larvae, helminth larvae extract, helminth larvae ESP, live helminth eggs, ground helminth eggs, helminth eggs extract, and helminth eggs ESP. The helminth compound may comprise a helminth selected from the group of helminths that do not naturally colonize humans and are otherwise non-pathogenic to humans, but through a repeated transitory infection in the gastorintestinal mucosa or the simulation of the same, offer an immunological benefit to an asthmatic, allergic and or hypersensitive individual.

The helminth compound will simulate an infection in the human, and, in doing so, stimulate the immune system in a way in which it may protect allergic humans from the inappropriate immune response associated with allergies and asthma. This stimulation is maintained by repeated administration of the helminth compound. The helminth compound is made from the group of helminths that colonize other animals, but not in humans, and has no associated pathology in humans. The helminth compound derived from these groups will establish only a transient infection in the human or will simulate the same, and, in doing so, stimulate the immune system in a way in which it may protect allergic humans from the inappropriate immune response associated with allergies and asthma. This stimulation is maintained by repeating the transient infection or simulation of a parasitic helminth infection with the helminth compound. In the preferred embodiment of the invention, the helminth to be used is Haemonchus contortus (H. contortus), or ESP therefrom, which will, as further described below, as a third stage juvenile larvae, locate itself temporarily in the stomach mucosa of humans who ingest it or ESP, will simulate a parasitic helminth infection.

H. contortus is a nematode that infects small ruminants. It releases a variety of ESP into the host and, although the composition of ESP is largely unknown, it is able to induce up to 90% protection from the host's immune response to eliminate it (A. Yatsuda. Comprehensive analysis of the secreted proteins of the parasite Haemonchus contortus reveals extensive sequence variation and differential immune recognition).

The invention thus relates to a method of treating, a therapy or prophylaxis of allergic and other IgE-mediated disorders, including, but not limited to, asthma, allergies, specifically, common food allergies, hypersensitivity and anaphylactic reactions, which are marked by an inappropriate IgE immune response including an aberrant and or enhanced IgE antibody production to benign antigens, with a non-human colonizing helminth compound, on a repetitive basis and in an amount sufficient to establish a repeated transitory gastrointestinal infection and thereby stimulating a regulatory immune response characterized by the production of Th2, TREG and certain cytokines, including, but not limited to IL-10, to eliminate or ameliorate the inappropriate immune response in an asthmatic, allergic and or hypersensitive individual.

An embodiment of the present invention describes using, on a repetitive basis, a non-human colonizing helminth compound, in an amount sufficient to establish a transitory parasitic helminth infection and or to simulate in a parasitic helminth infection, thereby having immunosuppressive effect against benign antigens and or stimulating a regulatory immune response as a therapy or prophylaxis of allergy and other IgE-mediated disorders. In doing so, the administration of the helminth compound will reverse or ameliorate the allergic sensitivity, thereby also reversing or ameliorating other behavioral and physical conditions, specifically autism and stuttering.

The helminth compound may comprise or be derived from H. contortus. Also, the helminth compound may comprise or be derived from the group of helminths from the families of Ostertagia, Trichostrongylus, Trichostrongylus, Bunostomum, Nematodiriasis, Oesophagostomum, Trichuriasis and Chabertia.

The invention further relates to a pharmaceutical composition comprising a helminth compound and a pharmaceutically acceptable carrier.

In addition, the invention relates to a method of producing and or manufacturing a helminth compound comprising isolating a helminth from the stool of a prepatory animal, cleaning it, maintaining it in a specific pathogen-free environment, and formulating a pharmaceutical composition and a pharmaceutically acceptable carrier.

In addition, the invention relates to a method of producing and or manufacturing a helminth compound comprising helminth ESP cultivated from an adult helminth, helminth larvae, and or helminth eggs, cleaning it, maintaining it in a specific pathogen-free environment, and formulating a pharmaceutical composition and a pharmaceutically acceptable carrier.

### BRIEF DESCRIPTION OF THE DRAWINGS

FIG. 1 is a photograph illustrating the RT-PCR detection of β-actin (βA), TGF-β (T), and IL-10 (10) transcripts from abomasal lymph node lymphocytes of two lambs infected with H. contortus.

### DETAILED DESCRIPTION OF THE PREFERRED EMBODIMENTS

The present invention describes using, on a repetitive basis, a non-human colonizing helminth compound, in an amount sufficient to establish a transitory infection or simulate a helminth infection and thereby having immunosuppressive effect against benign antigens by blocking the induction of allergen-specific IgE and or stimulating a regulatory immune response characterized by the production of T helper cells 2 (Th2), T regulatory helper cells (TREG) and certain cytokines, including, but not limited to interleukin 10 (IL-10), as a therapy or prophylaxis of allergy and other IgE-mediated disorders, which are marked by an inappropriate IgE immune response including, but not limited to an aberrant and or enhanced IgE antibody production to benign antigens. The invention presents using helminth compound by administering it in a frequency and amount sufficient to eliminate or ameliorate the inappropriate immune response in an asthmatic and or allergic individual. This invention is generally directed to diseases IgE-mediated disorders, including asthma, allergies, hypersensitivity and anaphylactic reactions. More specifically, the present invention is directed toward the treatment of CFA. While the present invention discloses specific information about the treatment of CFA, the disclosure is in no way limiting to CFA.

Allergic individuals (or hypersensitve individuals) who are exposed to allergens, such as common food products, will react in an allergic manner otherwise described herein. Further, the exposure to these allergens can also result the manifestation of other diseases, specifically the diseases of autism and stuttering. There is growing evidence that many people with autism are sensitive to certain food products. Hence, the present invention describes using, on a repetitive basis, a non-human colonizing helminth compound, in an amount sufficient to establish a transitory parasitic helminth infection and or to simulate in a parasitic helminth infection, thereby having immunosuppressive effect against benign antigens and or stimulating a regulatory immune response as a therapy or prophylaxis of allergy and other IgE-mediated disorders. In doing so, the administration of the helminth compound will reverse or ameliorate the allergic sensitivity, thereby also reversing or ameliorating other behavioral and physical conditions, specifically autism and stuttering.

Certain Parasitic Helminths that Infect Ruminants are Useful

Some embodiments of the present invention are based upon the recognition that diseases that involve hypersensitivity and anaphylactic reactions, which are marked by an excessive IgE immune response including an aberrant/enhanced IgE antibody production to benign antigens, are treatable by the administration of a helminthic parasite compound preparation that will provide a method of creating an immune environment that is conducive to ameliorating or reducing IgE-mediated disorders, including asthma, food allergies, hypersensitivity and anaphylactic reactions or vaccinating an individual against IgE-mediated disorders, including asthma, food allergies, hypersensitivity and anaphylactic reactions.

In a preferred embodiment of the invention, the helminth compound is from a helminth that naturally colonizes in sheep because they are species specific. In other words, these parasites only affect ruminants, such as sheep, and cannot be passed to humans or other monogastric animals. When introduced into a human host, it is unable to reproduce or migrate from one host to another.

The present invention contemplates using the Haemonchus contortus (H. contortus) third stage larvae (L3), to comprise the helminth compound. H. contortus has a very short life cycle in sheep and is located in the stomach glands (Scientific name: Haemonchus contortus; Common name: Barber pole worm; Class: Sexemetea; Subclass: Rhabdita; Order: Stongylida; Superfamily: Trichostrongyloidea; Family: Trichostrongylidea). Natural hosts for H. contortus are sheep, goats, cattle, and wild ruminants. H. contortus is more prevalent in warm moist regions than in cold, dry ones. No intermediate host is required during its life cycle. Adult male and female worms live in the abomasum (or true stomach) of ruminant animals. The female deposits 5,000 to 10,000 eggs per day which pass out of the host with the feces. First stage juveniles hatch from the eggs. First and second stage juveniles feed on bacteria. Third stage juveniles retain the second stage cuticle as a sheath. Third stage juveniles do not feed and are infective for the vertebrate host. The ruminant becomes infected while grazing by eating the third-stage juveniles. Exsheathment occurs in the rumen, anterior to the abomasum, and the young worms pass into the abomasum where they burrow into the mucosa. Here they undergo another molt, and the fourth-stage juveniles come back into the paramucosal lumen of the abomasum. They begin to feed and undergo another molt before reaching adulthood. Mating of adults occurs and egg production commences. The eggs hatch in soil or water and develop directly to infective third-stage juveniles. Enormous numbers of juveniles may accumulate on heavily grazed pastures. The family contains many genera and species.

The males are 10 to 20 mm and the females 18 to 30 mm long. The white uteri and ovaries winding around the red blood-filled intestine give a twisted or barberpole appearance. The small buccal capsule bears a curved dorsal tooth. There are two prominent lateral spike-like cervical papillae near the junction of the first and second quarters of the esophagus. The male bursa has long lateral lobes and slender rays with a flap-like dorsal lobe located asymmetrically near the base of the left lateral lobe. The spicules are 450 to 500 um long, each with a terminal barb; the gubernaculum is navicular. Usually, the vulva is covered by an anterior thumb-like flap which may be reduced to a mere knob in some individuals. The oval eggs are 70 to 85 um long by 41 to 44 um wide and in the early stages of cleavage when laid. They are somewhat yellowish.

The present invention contemplates using several other ruminant helminths from the families of Ostertagia and Trichostrongylus, which are found in the stomachs of sheep, are also contemplated by the present invention to comprise the helminth compound candidates. Specifically, several species of Trichostrongylus are found in the small intestines. Sheep hookworms (Bunostomum) are found in the small intestine. Roundworms (Nematodiriasis) are also found in the small intestine. Another internal parasite of sheep found in the small intestine is the nodular worm (Oesophagostomum). Whipworms (Trichuriasis) and large mouth bowel worms (Chabertia) are found in the large intestines.

### Mechanism of Action

Recently, elevations of anti-inflammatory cytokines, such as interleukin-10 (IL-10) that occur during long-term helminth infections have been shown to be inversely correlated with allergic disease such as asthma and allergy. Thus, the induction of a robust anti-inflammatory regulatory network by the persistent immune challenge created by a parasitic helminth offers an explanation for the observed inverse association of many helminth infections with allergic disease. Stated another way, the failure to be infected with helminths predisposes an individual to allergic disease, such as asthma and allergies. Therefore, as further discussed below, a repeated transitory infection of the GI tract or the simulation of a parasitic helminth infection with ESP with the certain non-pathogenic helminths may offer a means of preventing or treating IgE-mediated disorders, including asthma, CFA, hypersensitivity and anaphylactic reactions.

Helminths incite an intestinal Th2 response, which can cause worm expulsion or limit the magnitude of infection. Employing immunosuppressive defenses, many helminth species survive for years within the gut, binary tree or mesenteric veins making thousands of eggs daily. Thus, these worms and/or their ova release molecules that reside the intestinal mucosal surface for years, inciting Th2-type inflammation. Infestation with helminths, which induce Th2-type inflammation, can decrease excessive immune response to unrelated allergens because they may cause the secretion of T helper cells 2 (Th2), T regulatory helper cells (TREG) and certain cytokines, including, but not limited to interleukin 10 (IL-10).

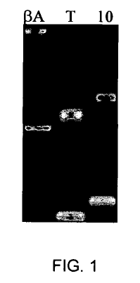

An intestinal homogenate of H. contortus, a surrogate for H. contortus ESP, has been shown to stimulate the expression of IL-10 in abomasal lymph node lymphocytes taken from H. contortus infected lambs when stimulated with H. contortus antigen, i.e., intestinal homogenate of H. contortus. As shown in FIG. 1, RT-PCR detection of β-actin (βA), TGF-β (T), and IL-10 (10) transcripts from abomasal lymph node lymphocytes of two lambs infected with H. contortus. Lymphocyte cultures were stimulated with parasite antigens, i.e., intestinal homogenate of H. contortus as a surrogate for H. contortus ESP, and collected four days later for RNA extraction. Six out of six infected lambs produced results similar to either the top or bottom panel.

As a means of simulating a chronic helminth infestation of humans, the present invention relates to using, on a repetitive basis, a non-human colonizing helminth compound, in an amount sufficient to establish a transitory infection or simulating a parasitic helminth infection and thereby having immunosuppressive effect against benign antigens by blocking the induction of allergen-specific IgE and or stimulating a regulatory immune response characterized by the production of Th2, TREG and certain cytokines, including, but not limited to IL-10. The initial or primary helminth infection is by a helminth compound that is only capable of making a transitory infection in humans or, with respect to ESP, simulating the same. In doing so, the immune response is the same as or similar to that of the immune response to a pathogenic parasite. In the case where the helminth compound is a live helminth, because humans are not the helminth compound's natural host, the parasite is unable to establish more than a transitory infection, dies, and is expelled from the body. In order to simulate a chronic parasitical infection, the administration of the non-human colonizing helminth compound is repeated until the disease symptoms abate.

### Helminth Compounds to be Used

It is anticipated that invisible, microscopically small live H. contortus ensheathed or exsheathed third stage juvenile larvae (L3) will provide the most profound Th2 mucosal conditioning because of their ability to make a transient infection in the human stomach mucosa and because of their ability to release a variety of ESP into the (sheep) host that is able to induce up to 90% protection from the host's immune response to eliminate it.

In a preferred embodiment of the present invention, it is contemplated that L3 will establish an infection in humans, but that infection should be transient, to avoid complications that may arise from a prolonged infection (Transient H. contortus infection has been established in Mongolian gerbils, with the strongest establishment in immunocompromised gerbils. The infection was expelled over time and fully mature (reproductively competent) worms were not observed. In some experiments with immunosuppressed mice, H. contortus did transiently infect the immunosuppressed mice. Mice and Mongolian gerbils, like humans, are monogastric). It is anticipated that the sheep, as the preparatory animal, will be raised in a pathogen-free environment according to methods known in the art and infected with H. contortus. The sheep will be tested to ensure the absence of human bacterial, mycobacterial, and viral pathogens and may need to be treated with: i) immunosuppressive glucocorticoids or azathioprine; ii) agents that impede Th2 effects like anti-histamines, anti-cytokines, or recombinant cytokines; and agents that influence intestinal moffiity like anti-cholinergics or opiates. Sheep will be tested to ensure a genetic background that renders them resistant to scrapie (a spongiform encephalopathy). The sheep's diet will be altered to reduce coarse fiber content and oral purgative to induce defecation. The female H. contortus burrowing in the abomasum (or true stomach) of the sheep will deposit 5,000 to 10,000 eggs per day, which pass out of the sheep with the feces. The oval eggs are 70 to 85 um long by 41 to 44 um wide and in the early stages of cleavage when laid. They are somewhat yellowish. The stool is collected and enzymatically digested to free the H. contortus eggs. The eggs are then isolated from liquefied stool by flotation on density gradients, screen filtration, Visser filtration, or centrifugal elutriation. Processed to render those bacteria and virus free, the eggs then require a maturation phase and are incubated under optimal conditions to mature the embryo, or hatch the egg and provide larval forms. Specifically, first stage juveniles hatch from the eggs. First and second stage juveniles feed on bacteria in a pathogen-free environment.

L3 will retain the second stage cuticle as a sheath, will not feed and are ready to establish a transitory infection in individuals in need of treatment. It will be refrigerated at between 45 degrees and 50 degrees Fahrenheit. The males are 10 to 20 mm and the females 18 to 30 mm long. The white uteri and ovaries winding around the red blood-filled intestine give a twisted or barberpole appearance. The small buccal capsule bears a curved dorsal tooth. There are two prominent lateral spike-like cervical papillae near the junction of the first and second quarters of the esophagus. The male bursa has long lateral lobes and slender rays with a flap-like dorsal lobe located asymmetrically near the base of the left lateral lobe. The spicules are 450 to 500 um long, each with a terminal barb; the gubernaculum is navicular. Usually, an anterior thumb-like flap that may be reduced to a mere knob in some individuals covers the vulva.

It is anticipated that individuals with asthma and allergies receive L3 added to a liquid, orally ingested, L3 will survive in body after being swallowed and will establish an infection in the individual, which will transient to avoid the complications that may arise from a prolonged infection. In order to avoid any egg production in the individual, the invention anticipates that L3 may only be comprised of males. Males and females may be separated by flotation on density gradients, screen filtration, Visser filtration, or centrifugal elutriation. It is anticipated that an amount ranging from about 1,000 to about 10,000 L3 will deposit themselves and establish a transitory infection in the mucosa of the stomach using the curved dorsal tooth. Because they are in the wrong host, L3 will die before they reach the forth stage of development and are capable of reproduction. Dead L3 will loosen from the stomach mucosa and will be eliminated completely during bowel movement. L3 will not be visible in the stool. It is anticipated that individuals with asthma and or allergies will repetitively drink doses of L3 at intervals of 7 to 14 days in order to simulate an infestation of the parasites.

The helminth compound of the invention will be formulated for oral dosage, at an acceptable pH with conventional fillers, carriers, and excipients known in the art, or presented in water. The amount of parasite administered to the individual in need thereof is an amount sufficient to prevent or treat the disease, which may vary depending upon the disease being treated or prevented, but is anticipated to range from about 1,000 to about 10,000 L3. In order to show the efficacy of the present invention, the TREG, Th2, cytokine productions, antigen-specific IgE, and assessment of anaphylactic symptom response must be distinguished.

There are several well-established disease activity indices that monitor clinical parameters, including evaluating the allergic response following challenge with the CFA, as well as laboratory and histological criteria. Using these indices, disease activity in the individual is monitored and evaluating. In the absence of disease symptoms, L3 treatment will be discontinued. Upon the return of disease symptoms, L3 treatment will resume.

In another preferred embodiment of the present invention, it is contemplated that the helminth compound will be ESP and will, by simulating the establishment of a parasitical helminth infection in humans, have an immunosuppressive effect against benign antigens by blocking the induction of allergen-specific IgE or stimulating a regulatory immune response characterized by the production of T helper cells 2 (Th2), T regulatory helper cells (TREG) and certain cytokines, including, but not limited to interleukin 10 (IL-10). In this embodiment, H. contortus will become established in sheep as described above. The sheep will be sacrificed and approximately 10,000 adult H. contortus worms will be harvested. Specifically, adult H. contortus worms will be collected at day 6 post infection and cultured for 7 days in RPMI 1640 with 100 U/ml penicillin, 10 Ig/mI streptomycin and 1% glucose. Supernatants will be collected at 48-hour intervals from days 1 through 7, pooled and concentrated to 1 mg/ml. The ESP will be treated for 15 minutes at 95C. to yield hiESP. Potential endotoxin contamination is neutralized by pre-incubation with 20 Ig/mI polymyxin B sulfate (Sigma) at 37C. for 30 minutes (A. Balic, et al. Eur. J. Immunol. 2004. 34: 3047-3059).

The entire disclosure of each of the cited literature references is incorporated herein by reference thereto.

It should be understood that the foregoing detailed description is provided for clarity only and is merely exemplary. The spirit and scope of the present invention are not limited to the above examples, but are encompassed by the claims.

What is claimed is:

1. A method of treating a disorder characterized by an increase in the level of IgE comprising administering to a mammal in need thereof, a therapeutically effective amount of excretory and secretory products from a helminth.

2. The method of claim 1 wherein said disorder is asthma.

3. The method of claim 1 wherein said disorder is an allergy.

4. The method of claim 3 wherein said allergy is a common food allergy.

5. The method of claim 1 wherein said disorder is autism.

6. The method of claim 1 wherein said disorder is stuttering.

7. The method of claim 1 wherein the excretory and secretory products from a helminth are selected from the helminth families consisting of Ostertagia, Trichostrongylus, Trichostrongylus, Bunostomum, Nematodiriasis, Oesophagostomum, Trichuriasis and Chabertia.

8. The method of claim 1 wherein the helminth excretory and secretory products are from the helminth Haemonchus contortus.

9. The method of claim 1 wherein said excretory and secretory products are isolated from the group consisting of live adult helminth, ground adult helminth, adult helminth extract, adult helminth ESP, live helminth larvae, ground helminth larvae, helminth larvae extract, helminth larvae ESP, live helminth eggs, ground helminth eggs, helminth eggs extract, and helminth eggs ESP.

10. The method of claim 1 wherein said excretory and secretory products are present in an intestinal homogenate of a helminth.

11. The method of claim 1 wherein said administering to a mammal in need thereof a therapeutically effective amount of excretory and secretory products from a helminth, stimulates the production of IL-10 in the mammal.

12. A method of treating a disorder characterized by an increase in the level of IgE comprising administering to a mammal in need thereof, a therapeutically effective amount of excretory and secretory products from a helminth, wherein administering the excretory and secretory products stimulates the level of IL-10 in the mammal.

13. The method of claim 12 wherein said excretory and secretory products are present in an intestinal homogenate of a helminth.

14. The method of claim 12 wherein said excretory and secretory products are present in an intestinal homogenate of Haemonchus contortus.

15. The method of claim 12 wherein said excretory and secretory products are isolated from cutures of live helminths.

16. The method of claim 12 wherein said excretory and secretory products are isolated from live cultures of Haemonchus contortus.

17. A method for producing a pharmaceutical composition comprising a helminth parasite preparation comprising the steps of:
(1) raising a preparatory animal in a specific human pathogen-free environment;
(2) isolating a helminth parasite from said preparatory animal to form a helminth parasite isolate;
(3) isolating excretory and secretory products from said helminth parasite isolate; and
(3) mixing the excretory and secretory products from step (3) with a pharmaceutically acceptable carrier.

18. The method of claim 17 wherein said parasite is Haemonchus contortus.
